(19) United States
(12) Patent Application Publication
(10) Pub. No.: US 2024/0252696 A1
(43) Pub. Date: Aug. 1, 2024

(54) TARGETING THE CHOLECYSTOKININ-B RECEPTOR FOR IMAGING AND EARLY DETECTION OF PANCREATIC CANCER AND PRE-CANCEROUS LESIONS

(71) Applicants: Georgetown University, Washington, DC (US); The USA as represented by the Secretary, Department of Health and Human Services, Bethesda, MD (US)

(72) Inventors: Jill P. Smith, Camp Hill, PA (US); Stephan Stern, Frederick, MD (US)

(73) Assignees: Georgetown University, Washington, DC (US); The USA as represented by the Secretary, Department of Health and Human Services, Bethesda, MD (US)

(21) Appl. No.: 17/925,582

(22) PCT Filed: May 26, 2021
(86) PCT No.: PCT/US2021/034339

§ 371 Date: Nov. 15, 2022

Related U.S. Application Data
(60) Provisional application No. 63/030,815, filed on May 27, 2020.

(51) Int. Cl. A61K 51/08; A61K 49/00

(52) U.S. Cl. CPC A61K 51/088; A61K 49/0056

(57) ABSTRACT

A method that includes detecting the presence of a pancreatic intraepithelial neoplasia lesion in a subject in vivo comprising administering to the subject a construct, or a pharmaceutically acceptable salt thereof, wherein the construct comprises:
(a) a polyethylene glycol-block-poly(L-lysine) polymer moiety, wherein the polyethylene glycol is thiol-functionalized;
(b) a cholecystokinin-B (CCK-B) receptor ligand coupled to the polyethylene glycol of the polymer moiety; and
(c) a detectable moiety complexed with, or conjugated to, the poly(L-lysine) of the polymer moiety,
wherein the construct is neutralized.

This application claims the benefit of U.S. Provisional Application No. 63/030,815, filed May 27, 2020, which is incorporated herein by reference.

### BACKGROUND

Pancreatic ductal adenocarcinoma (PDAC) has a dismal prognosis and with the current chemotherapeutic regimens the 5-year survival is only about 10%. Reasons for the poor outcome in pancreatic cancer includes the inability to diagnose pancreatic cancer in early or precancerous stages, its propensity to metastasize rapidly, and the lack of available targeted therapies. Computerized tomography (CT) and magnetic resonance imaging (MRI) are recommended for diagnosing PDAC, and these imaging techniques are widely used when PDAC is suspected. However, these imaging techniques are limited to detecting tumors greater than 2 cm and 23% of small lesions cannot be detected. One approach that has been used in an attempt to detect pancreatic cancer in ‘earlier’ stages is endoscopic ultrasound (EUS). EUS is very sensitive for evaluating pancreatic cysts; however, the overwhelming majority of cysts are benign. Furthermore, only about 15% of pancreatic cancers arise from cysts; the majority (85%) of pancreatic cancers develop from a microscopic precursor lesion called pancreatic intraepithelial neoplasia (PanINs). PanINs are not identified by the standard radiographic imaging or endoscopic techniques.

Early stages of disease leading to the development of pancreatic carcinoma have been difficult to study in human subjects since most are diagnosed in the late stages of disease. A genetically engineered murine animal model that has conditional expression of an endogenous KRAS (G12D) allele in murine embryo has been established and researchers have used this model and variations of this model to study pancreatic carcinogenesis. In the examples disclosed herein a variant of this mutant KRAS murine model, LSL-Kras^{G12D/+}, was used; P48-Cre, that highly resembles pathogenesis in the precancerous human pancreas in that it progresses through advancing grades of premalignant lesions (PanINs 1, 2, and 3), allowing investigators to study potential biomarkers associated with pancreatic carcinogenesis and early stage malignancy.

### SUMMARY

Disclosed herein is a method comprising detecting the presence of a pancreatic intraepithelial neoplasia lesion in a subject in vivo comprising administering to the subject a construct, or a pharmaceutically acceptable salt thereof, wherein the construct comprises:
(a) a polyethylene glycol-block-poly(L-lysine) polymer moiety, wherein the polyethylene glycol is thiol-functionalized;
(b) a cholecystokinin-B (CCK-B) receptor ligand coupled to the polyethylene glycol of the polymer moiety; and
(c) a detectable moiety complexed with, or conjugated to, the poly(L-lysine) of the polymer moiety,
wherein the construct is neutralized.

Also disclosed herein is a method for making a construct comprising:
(a) conjugating a maleimide-containing gastrin-10 peptide with a block copolymer resulting in a nanoparticle, the block copolymer comprising (i) a thiol-functionalized polyethylene glycol block and (ii) a poly(L-lysine) block; and
(b) mixing the resulting nanoparticle with at least one detectable moiety-tagged complexing agent or detectable moiety-tagged coupling agent.

Additionally disclosed herein is a method for making a construct comprising:
(a) conjugating a maleimide-containing gastrin-10 peptide with a block copolymer resulting in a targeted polymer construct, the block copolymer comprising (i) a thiol-functionalized polyethylene glycol block and (ii) a poly(L-lysine) block; and
(b) mixing the resulting targeted polymer construct with at least one detectable moiety-tagged complexing agent or detectable moiety-tagged coupling agent.

Further disclosed herein is a method comprising:
(I) detecting the presence of a pancreatic intraepithelial neoplasia lesion in a subject in vivo comprising administering to the subject a first construct, or a pharmaceutically acceptable salt thereof, wherein the first construct comprises:
(a) a polyethylene glycol-block-poly(L-lysine) polymer moiety, wherein the polyethylene glycol is thiol-functionalized;
(b) a cholecystokinin-B (CCK-B) receptor ligand coupled to the polyethylene glycol of the polymer moiety; and
(c) a detectable moiety complexed with, or conjugated to, the poly(L-lysine) of the polymer moiety,
wherein the first construct is neutralized, and
(II) in a subject in which a pancreatic intraepithelial neoplasia lesion is detected, administering to the subject a therapeutically effective amount of a second construct, or a pharmaceutically acceptable salt thereof, comprising:
(a) a polyethylene glycol-block-poly(L-lysine) polymer moiety, wherein the polyethylene glycol is thiol-functionalized;
(b) a cholecystokinin-B (CCK-B) receptor ligand coupled to the polyethylene glycol of the polymer moiety; and
(c) a therapeutically active agent complexed with the poly(L-lysine) of the polymer moiety,
wherein the second construct is neutralized.

The foregoing will become more apparent from the following detailed description, which proceeds with reference to the accompanying figures.

### BRIEF DESCRIPTION OF THE DRAWINGS

FIGS. 1A-1B. Synthesis of target-specific polyplex micelle nanoparticle (FIG. 1A). The gastrin-10 targeting ligand moiety is conjugated to polyethylene glycol (PEG) which in turn is conjugated to a poly L-lysine block chain rendering the backbone positively charged. The oligonucleotides were conjugated with either Alexa Fluor™ 488 or Alexa Fluor™ 647. (FIG. 1B) When the positively charged L-lysine co-polymer block complexes with the negatively charged fluorophore-tagged oligonucleotide, a self-assembled neutral polyplex micelle forms.

FIGS. 2A-2F. In vivo and ex-vivo imaging of LSL-Kras^{G12D/+}; P48-Cre (KC) mice 5 hrs. post injection of Alexa Fluor™ 647/488-labeled, CCK-BR-targeted polyplex nanoparticle. (FIG. 2A) Side view and (FIG. 2B) dorsal view of a representative anesthetized 5 month-old mouse imaged in the IVIS imaging system shows near infrared fluorescent signal in the area of the mouse pancreas, (FIG. 2C) Ex-vivo fluorescent imaging of major organs only shows significant fluorescence in the pancreas. (FIG. 2D) Optical ex-vivo imaging of the pancreas. (FIG. 2E) IVIS system ex-vivo imaging of pancreas shows intense fluorescence in the near infrared range, consistent with Alexa Fluor™ 647. (FIG. 2F) Epi-fluorescence scale showing range from low (brown) to high (Yellow) intensity.

FIGS. 3A-3D. Histology and Immunohistochemistry (IHC) of LSL-Kras^{G12D/+}; P48-Cre murine pancreas. (FIG. 3A) H&E histologic section of the 10-month old mouse pancreas reveals evidence of advanced PanIN lesions and fibrosis of the pancreas microenvironment. (FIG. 3B) The same pancreas section reacted with anti-Alexa488 antibody shows marked immunoreactivity consistent with polyplex micelle nanoparticle binding to the CCK-BR on advanced PanIN lesions. (FIG. 3C) IHC for anti-Alexa488 antibody shows the intensity of the anti-Alexa488 antibody staining is greatest in the PanIN-3 lesions in the pancreas of 5-month old mouse. The normal pancreatic acinar cells are void of staining. Bar=100 μm. (FIG. 3D) Selective immunoreactivity with intense staining in PanIN-3 lesions of the pancreas of a 5-month old KC mouse. Again, normal pancreatic acinar do not take up the staining and hence lack of binding from the targeted polyplex micelle nanoparticle. Bar=200 μm.

FIGS. 4A-4F. Immunohistochemistry for Alexa488 in various organs of mouse after injection of the CCK-BR targeted polyplex micelle nanoparticle. Immunoreactivity is negligible in other organs including (FIG. 4A) stomach, (FIG. 4B) spleen, (FIG. 4C) lung, (FIG. 4D) heart (FIG. 4E) liver, and (FIG. 4F) kidney. The arrow in the liver (FIG. 4E) shows trace staining for Alexa488 in a Kupffer cell. The arrow in the kidney (FIG. 4F) shows immunoreactivity from excretion of the polyplex in the luminal surfaces of the collecting ducts.

FIGS. 5A-5D. Immunoreactivity with PEG antibody in 5 month-old KRAS mouse pancreas. (FIG. 5A) Pancreas from a mouse treated with CCK-BR targeted polyplex nanoparticles confirms PEG staining in the PanINs. (FIG. 5B). A PanIN lesion in pancreas of a 5-month old mouse treated with targeted polyplex nanoparticles shows immunoreactivity for PEG while adjacent normal acinar cells in pancreas tissue are negative. (FIG. 5C) Negative immunoreactivity to PEG antibody in PanINs of 5 month old KRAS mice injected with untargeted polyplex nanoparticle (20×). (FIG. 5D) Image from a 5-month old KRAS mouse pancreas injected with untargeted polyplex nanoparticle is negative for immunoreactivity for PEG.

FIGS. 6A-6B. Human pancreas tissue array (US Biomax, #PA481) reacted with CCK-B receptor goat polyclonal antibody (1:200; Abcam 77077). FIG. 6A. shows minimal to no staining in normal human pancreas. FIG. 6B: positive staining in human pancreas with PanINs and cancer.

### SEQUENCE LISTING

The nucleic acid sequences listed in the accompanying sequence listing are shown using standard letter abbreviations for nucleotide bases as defined in 37 C.F.R. 1.822. Only one strand of each nucleic acid sequence is shown, but the complementary strand is understood as included by any reference to the displayed strand. The Sequence Listing is submitted as an ASCII text file, created on May 26, 2021, which is incorporated by reference herein.

### DETAILED DESCRIPTION

### Terminology

The following explanations of terms and methods are provided to better describe the present compounds, compositions and methods, and to guide those of ordinary skill in the art in the practice of the present disclosure. It is also to be understood that the terminology used in the disclosure is for the purpose of describing particular embodiments and examples only and is not intended to be limiting.

“Administration” as used herein is inclusive of administration by another person to the subject or self-administration by the subject.

An “animal” refers to living multi-cellular vertebrate organisms, a category that includes, for example, mammals and birds. The term mammal includes both human and non-human mammals. Similarly, the term “subject” includes both human and non-human subjects, including birds and non-human mammals, such as non-human primates, companion animals (such as dogs and cats), livestock (such as pigs, sheep, cows), as well as non-domesticated animals, such as the big cats. The term subject applies regardless of the stage in the organism's life cycle. Thus, the term subject applies to an organism in utero or in ovo, depending on the organism (that is, whether the organism is a mammal or a bird, such as a domesticated or wild fowl).

The term “subject” includes both human and non-human subjects, including birds and non-human mammals, such as non-human primates, companion animals (such as dogs and cats), livestock (such as pigs, sheep, cows), as well as non-domesticated animals, such as the big cats. The term subject applies regardless of the stage in the organism's life cycle. Thus, the term subject applies to an organism in utero or in ovo, depending on the organism (that is, whether the organism is a mammal or a bird, such as a domesticated or wild fowl).

A “therapeutically effective amount” refers to a quantity of a specified agent sufficient to achieve a desired effect in a subject being treated with that agent. Ideally, a therapeutically effective amount of an agent is an amount sufficient to inhibit or treat the disease or condition without causing a substantial cytotoxic effect in the subject. The therapeutically effective amount of an agent will be dependent on the subject being treated, the severity of the affliction, and the manner of administration of the therapeutic composition.

“Treatment” refers to a therapeutic intervention that ameliorates a sign or symptom of a disease or pathological condition after it has begun to develop, or administering a compound or composition to a subject who does not exhibit signs of a disease or exhibits only early signs for the purpose of decreasing the risk of developing a pathology or condition, or diminishing the severity of a pathology or condition. As used herein, the term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease. The phrase “treating a disease” refers to inhibiting the full development of a disease, for example, in a subject who is at risk for a disease such as diabetes. “Preventing” a disease or condition refers to prophylactic administering a composition to a subject who does not exhibit signs of a disease or exhibits only early signs for the purpose of decreasing the risk of developing a pathology or condition, or diminishing the severity of a pathology or condition.

“Pharmaceutical compositions” are compositions that include an amount (for example, a unit dosage) of one or more of the disclosed compounds together with one or more non-toxic pharmaceutically acceptable additives, including carriers, diluents, and/or adjuvants, and optionally other biologically active ingredients. Such pharmaceutical compositions can be prepared by standard pharmaceutical formulation techniques such as those disclosed in Remington's Pharmaceutical Sciences, Mack Publishing Co., Easton, PA (19th Edition).

The terms “pharmaceutically acceptable salt or ester” refers to salts or esters prepared by conventional means that include salts, e.g., of inorganic and organic acids, including but not limited to hydrochloric acid, hydrobromic acid, sulfuric acid, phosphoric acid, methanesulfonic acid, ethanesulfonic acid, malic acid, acetic acid, oxalic acid, tartaric acid, citric acid, lactic acid, fumaric acid, succinic acid, maleic acid, salicylic acid, benzoic acid, phenylacetic acid, mandelic acid and the like. “Pharmaceutically acceptable salts” of the presently disclosed compounds also include those formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)aminomethane, and tetramethylammonium hydroxide. These salts may be prepared by standard procedures, for example by reacting the free acid with a suitable organic or inorganic base. Any chemical compound recited in this specification may alternatively be administered as a pharmaceutically acceptable salt thereof. “Pharmaceutically acceptable salts” are also inclusive of the free acid, base, and zwitterionic forms. Descriptions of suitable pharmaceutically acceptable salts can be found in Handbook of Pharmaceutical Salts, Properties, Selection and Use, Wiley VCH (2002). When compounds disclosed herein include an acidic function such as a carboxy group, then suitable pharmaceutically acceptable cation pairs for the carboxy group are well known to those skilled in the art and include alkaline, alkaline earth, ammonium, quaternary ammonium cations and the like. Such salts are known to those of skill in the art. For additional examples of “pharmacologically acceptable salts,” see Berge et al., J. Pharm. Sci. 66:1 (1977).

“Pharmaceutically acceptable esters” includes those derived from compounds described herein that are modified to include a carboxyl group. An in vivo hydrolysable ester is an ester, which is hydrolysed in the human or animal body to produce the parent acid or alcohol. Representative esters thus include carboxylic acid esters in which the non-carbonyl moiety of the carboxylic acid portion of the ester grouping is selected from straight or branched chain alkyl (for example, methyl, n-propyl, t-butyl, or n-butyl), cycloalkyl, alkoxyalkyl (for example, methoxymethyl), aralkyl (for example benzyl), aryloxyalkyl (for example, phenoxymethyl), aryl (for example, phenyl, optionally substituted by, for example, halogen, C.sub.1-4 alkyl, or C.sub.1-4 alkoxy) or amino); sulphonate esters, such as alkyl- or aralkylsulphonyl (for example, methanesulphonyl); or amino acid esters (for example, L-valyl or L-isoleucyl). A “pharmaceutically acceptable ester” also includes inorganic esters such as mono-, di-, or tri-phosphate esters. In such esters, unless otherwise specified, any alkyl moiety present advantageously contains from 1 to 18 carbon atoms, particularly from 1 to 6 carbon atoms, more particularly from 1 to 4 carbon atoms. Any cycloalkyl moiety present in such esters advantageously contains from 3 to 6 carbon atoms. Any aryl moiety present in such esters advantageously comprises a phenyl group, optionally substituted as shown in the definition of carbocycylyl above. Pharmaceutically acceptable esters thus include C₁-C₂₂ fatty acid esters, such as acetyl, t-butyl or long chain straight or branched unsaturated or omega-6 monounsaturated fatty acids such as palmoyl, stearoyl and the like. Alternative aryl or heteroaryl esters include benzoyl, pyridylmethyloyl and the like any of which may be substituted, as defined in carbocyclyl above. Additional pharmaceutically acceptable esters include aliphatic L-amino acid esters such as leucyl, isoleucyl and especially valyl.

For therapeutic use, salts of the compounds are those wherein the counter-ion is pharmaceutically acceptable. However, salts of acids and bases which are non-pharmaceutically acceptable may also find use, for example, in the preparation or purification of a pharmaceutically acceptable compound.

The pharmaceutically acceptable acid and base addition salts as mentioned hereinabove are meant to comprise the therapeutically active non-toxic acid and base addition salt forms which the compounds are able to form. The pharmaceutically acceptable acid addition salts can conveniently be obtained by treating the base form with such appropriate acid. Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic (i.e. hydroxybutanedioic acid), tartaric, citric, methanesulfonic, ethanesulfonic, benzenesulfonic, p-toluenesulfonic, cyclamic, salicylic, p-aminosalicylic, pamoic and the like acids. Conversely said salt forms can be converted by treatment with an appropriate base into the free base form.

The compounds containing an acidic proton may also be converted into their non-toxic metal or amine addition salt forms by treatment with appropriate organic and inorganic bases. Appropriate base salt forms comprise, for example, the ammonium salts, the alkali and earth alkaline metal salts, e.g. the lithium, sodium, potassium, magnesium, calcium salts and the like, salts with organic bases, e.g. the benzathine, N-methyl-D-glucamine, hydrabamine salts, and salts with amino acids such as, for example, arginine, lysine and the like.

The term “addition salt” as used hereinabove also comprises the solvates which the compounds described herein are able to form. Such solvates are for example hydrates, alcoholates and the like.

The term “quaternary amine” as used hereinbefore defines the quaternary ammonium salts which the compounds are able to form by reaction between a basic nitrogen of a compound and an appropriate quaternizing agent, such as, for example, an optionally substituted alkylhalide, arylhalide or arylalkylhalide, e.g. methyliodide or benzyliodide. Other reactants with good leaving groups may also be used, such as alkyl trifluoromethanesulfonates, alkyl methanesulfonates, and alkyl p-toluenesulfonates. A quaternary amine has a positively charged nitrogen. Pharmaceutically acceptable counterions include chloro, bromo, iodo, trifluoroacetate and acetate. The counterion of choice can be introduced using ion exchange resins.

### Overview

Survival from pancreatic cancer remains extremely poor in part because this malignancy is not diagnosed in the early stages and the precancerous lesions are not seen on routine imaging with computerized tomography, MRI, or endoscopic ultrasound. The cholecystokinin-B receptor (CCK-BR) becomes over-expressed in precancerous pancreatic intraductal neoplasia (PanIN) lesions of the pancreas. CCK-BR is not detected in normal mouse or human pancreas but becomes over-expressed in PanIN lesions and markedly over-expressed in PDAC.

Disclosed herein is a targeted polyplex nanoparticle that can be conjugated with a fluorophore or radioactive molecule (e.g., technetium or fluorine-18). In conjunction with an imaging device, such as SPECT-CT or PET-CT, the labeled, targeted polyplex nanoparticle can detect the presence of precancerous PanIN lesions. The CCK-BR-targeted polyplex nanoparticles do not cross react with other tissues.

One embodiment disclosed herein is a biodegradable fluorescent polyplex nanoparticle that selectively targets the CCK-BR. When administered to a genetically engineered mouse model of pancreatic carcinogenesis, the polyplex localizes only to the precancerous lesions and is detected by fluorescence in living mice. Ex vivo immunohistochemistry staining of the labeled polyplex with serial H&E histopathological evaluation confirmed localization to the PanIN lesions. This imaging tool can be developed for screening those at high risk for pancreatic cancer with the goal to improve survival with early detection.

Disclosed herein are block copolymer (polyethylene glycol-block-polylysine (PEG-b-PLL) polyplex micelle nanoparticles (polyplex NPs) that bind selectively to the CCK-receptor and deliver a payload of a detectable moiety to at least one PanIN lesion. In particular, the nanoparticles are linked to CCK receptor ligand (e.g., gastrin-10 peptide or a DNA aptamer) to the poly-L-lysine (PLL) of the nanoparticle through a short PEG segment using maleimide chemistry. The detectable moiety may be a fluorophore or a radioactive molecule. The detectable moiety may include a complexing or coupling moiety such as an oligonucleotide. The detectable moiety may be detected by detecting techniques such as imaging systems. In certain embodiments, the detectable moiety may be detected via in vivo imaging. The detectable moiety is added to the block copolymer construct by electrostatic complexation or direct conjugation. These form a micelle that is biodegradable and nontoxic.

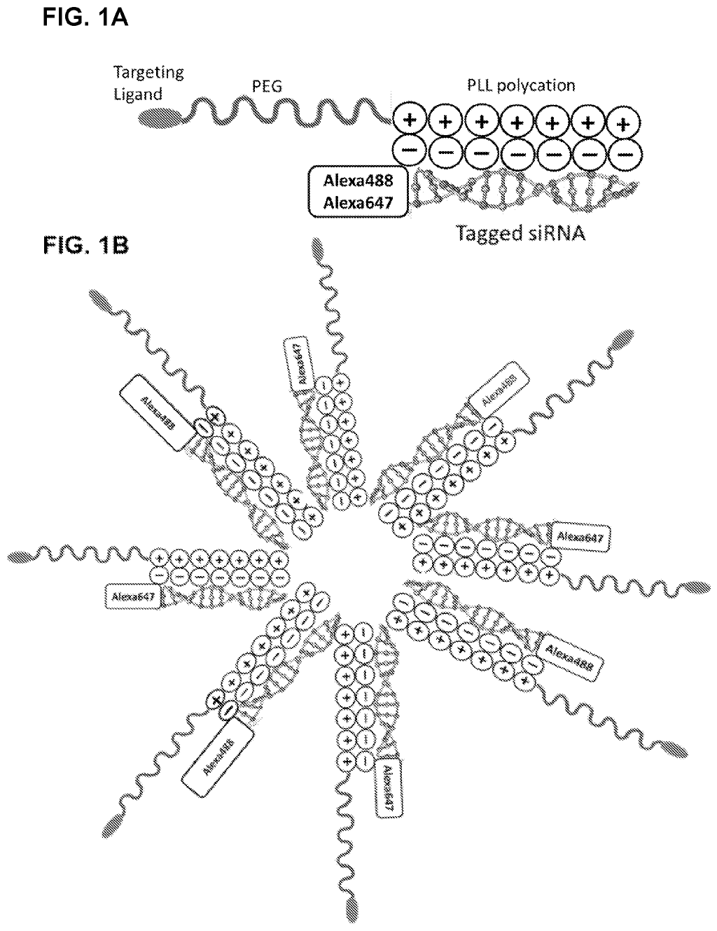

For example, disclosed herein is a polyplex micelle nanoparticle that targets the CCK-BR that includes a thiol functionalized polyethylene glycol-block-poly (L-lysine) (SH-PEG-PLL) that was rendered specific to the CCK-BR by conjugation of gastrin-10 peptide (Ga-10) polymer (FIG. 1A). When the backbone moiety was complexed with negatively charged oligonucleotides a self-assembled polyplex micelle forms (FIG. 1B). The polyplex nanoparticle may include two separate oligonucleotides: Custom RNA, 5′ Alexa Fluor™ 647 and 5′ Alexa Fluor™ 488 (Life Technologies, ThermoFisher Scientific). The Alexa Fluor™ 647 was selected for its fluorescent properties in the Near Infrared range that allows imaging of mutant KRAS mice with an IVIS Lumina III in Vivo Optical Imaging System. The Alexa Fluor™ 488 was also used in the polyplex micelle so that localization of the polyplex micelle in the animal tissues could be confirmed by immunohistochemistry in pancreas and other organs ex-vivo.

Disclosed herein are novel NPs to deliver a detectable moiety using a CCK-receptor-targeted polyethylene glycol-block-poly(L-lysine) (PEG-b-PLL) polyplex. The targeted PEG-b-PLL polyplexes was designed to contain three basic features: (i) a short cationic segment (PLL) for the complexation of a detectable moiety (e.g. a detectable moiety conjugated to an oligonucleotide (e.g., an RNA)) or direct conjugation to an imaging probe, (ii) a hydrophilic and biologically inert segment (PEG), and (iii) a cell surface targeting moiety (a peptide, gastrin-10). This block copolymer design will facilitate small polyplex formation following electrostatic interaction between the cationic polylysine moiety and negatively charged detectable moiety, resulting in charge neutralization and self-assembly into a polyplex structure with detectable moiety contained in the core surrounded by PEG conjugated to the targeting ligand gastrin-10 on the surface (FIG. 1B). In certain embodiments, the polyplex disclosed herein is in the form of a micelle. The conjugation of gastrin-10 to the PEG-b-PLL polymer is performed via maleimide-thiol coupling chemistry.

In particular, the block copolymer includes two block moieties: (1) thiol-functionalized polyethylene glycol (PEG); and (2) poly(L-lysine) (PLL). The block copolymer (referred to herein as “SH-PEG-PLL”) may have a structure represented by

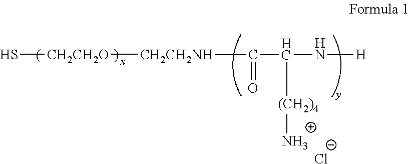

wherein x is 22 to 454, more particularly 45 to 275; and y is 10 to 100, more particularly 20 to 50. The number-average molecular weight of the PEG may range from 1000 Da to 20,000 Da. The number-average molecular weight of the PLL may range from 1600 Da to 16000 Da. In certain embodiments, x is 113 and y is 27, and the PEG molecular weight is 5000 g/mole (Da) and the PLL molecular weight is 4400 Da. In certain embodiments, 10 to 30%, more particularly about 20% of the PEG chains are thiol functionalized.

The maleimide-containing gastrin-10 peptide may have a structure of:

3-maleimido-propionyl-Glu-Glu-Glu-Ala-Tyr-Gly-Trp-Met-Asp-Phe-NH₂ (molecular formula: C₆₅H₇₉N₁₃O₂₂S; molecular weight: 1426.48 Da).
The maleimide-containing gastrin-10 peptide can be conjugated to any thiol (—SH) group through Michael addition to form a stable thioether bond.

The resulting targeted block copolymer has a structure of:

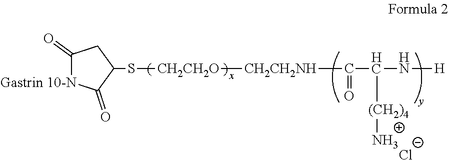

wherein x and y are the same as above.

In certain embodiments, the block copolymer construct together has a structure of:

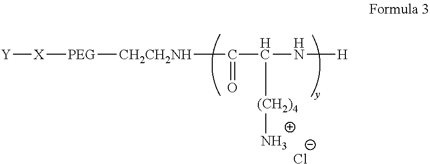

wherein Y is the cholecystokinin-B (CCK-B) receptor ligand; X is a linker; PEG is polyethylene glycol; and y is 10 to 200, more particularly 20 to 50.

The linker X may be a thioether or a group derived from a methoxy or carboxy linking agent.

At least one detectable moiety-tagged coupling or complexing agent (e.g., siRNA) is mixed with the block copolymer under conditions sufficient for electrostatically complexing the detectable moiety-tagged agent with the poly(L-lysine) of the polymer moiety. The detectable moiety-tagged agent may be a fluorophore labeled oligonucleotide or a radioactive labeled oligonucleotide (e.g., Technetium-99m for SPECT-CT imaging, F-18 for PET-CT imaging). Alternatively, the free amines of the polylysine portion of the block copolymer may be conjugated directly with a radioactive probe (e.g., F-18 trifluoroacetic anhydride for PET-CT imaging). For example, F-18-trifluoroacetic anhydride may be reacted with the amine(s) of the PEG-polylysine block co-polymer, to form a PEG-polylysine-F-18-trifluoroacetyl. In certain embodiments, only up to 10% of the amines are modified with the F-18-trifluoroacetic anhydride to allow for sufficient free amines remaining to complex with anionic RNA to form the polyplex micelle nanoparticle. The F-18-trifluoroacetic anhydride is not reacted with the targeted gastrin-PEG-polylysine, because the amine-end of the gastrin peptide and amine of the internal alanine would also be acetylated and lose targeting activity for binding the CCK-BR. In certain embodiments, the targeted polyplex only has 30% gastrin-PEG-polylysine, with the remaining 70% untargeted PEG-polylysine labeled with the F-18-trifluoroacetyl.

The relative concentrations of the block copolymer and the detectable moiety-tagged agent may vary. In certain embodiments, the relative concentrations are appropriate to provide N/P (nitrogen of polylysine amine (NH₂⁺) verses phosphate (PO₄⁻) of siRNA) of 0.5 to 20, more particularly 2 to 10.

In certain embodiments, the siRNA may have a structure of:

Sense:
(SEQ. ID No: 1)
AGCU-ACACUAUCGAGCAAUUAAC UU
and
Anti-Sense:
(SEQ. ID No: 2)
AAGUUAAUUGCUCGAUAGUGU-AGCU.

The cholecystokinin-B (CCK-B) receptor ligand may be gastrin-10 or a DNA aptamer as disclosed, for example, in Nucleic Acid Ther. 2017 Feb. 1; 27(1):23-35). An illustrative DNA aptamer has a structure of:

(SEQ. ID No: 3)
CATGGTGCAG GTGTGGCTGG GATTCATTTG CCGGTGCTGG
TGCGTCCGCG GCCGCTAATC CTGTTC.

The nanoparticles disclosed herein are biodegradable and biocompatible. Furthermore, when assembled, the nanoparticles will protect the detectable moiety from degradation in the NP core.

The detectable moiety may be a fluorophore. A fluorophore is a functional group, or portion, of a molecule that causes the molecule to fluoresce when exposed to an excitation source. In certain embodiments, the fluorophore is a fluorescent compound such as a dye. For example, the dye may be a sulfonate dye such as a sulfonated coumarin, a sulfonated rhodamine, a sulfonated cyanine, or a sulfonated xanthene. Illustrative fluorophores include the Alex Fluor® class of compounds. Structures of several Alex Fluor® compounds are shown below.

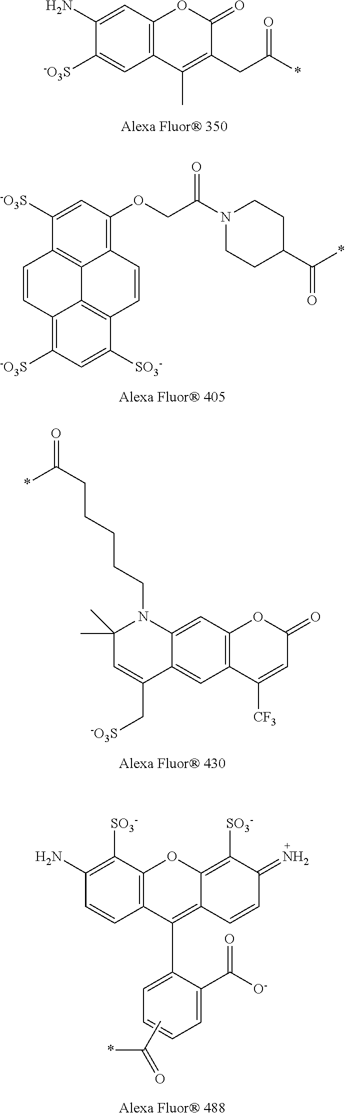

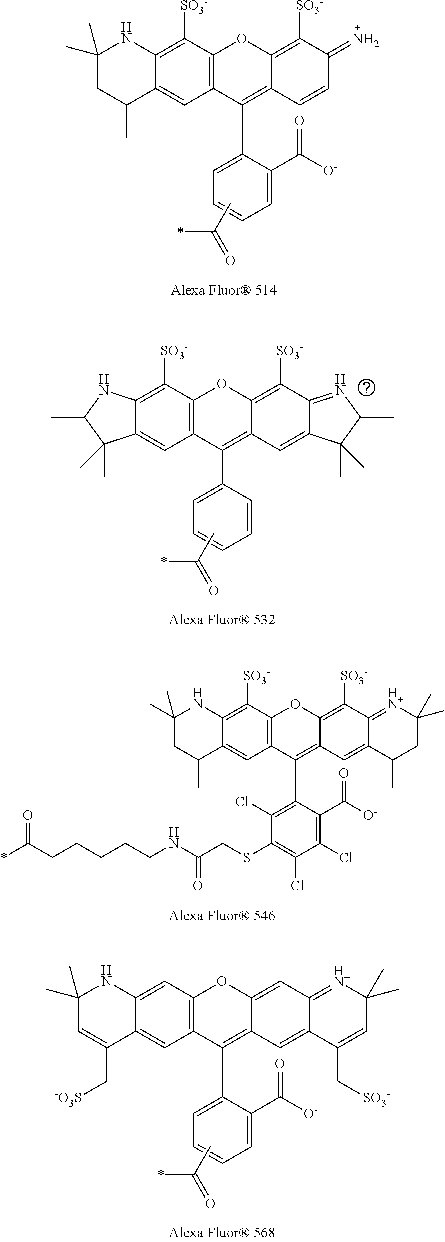

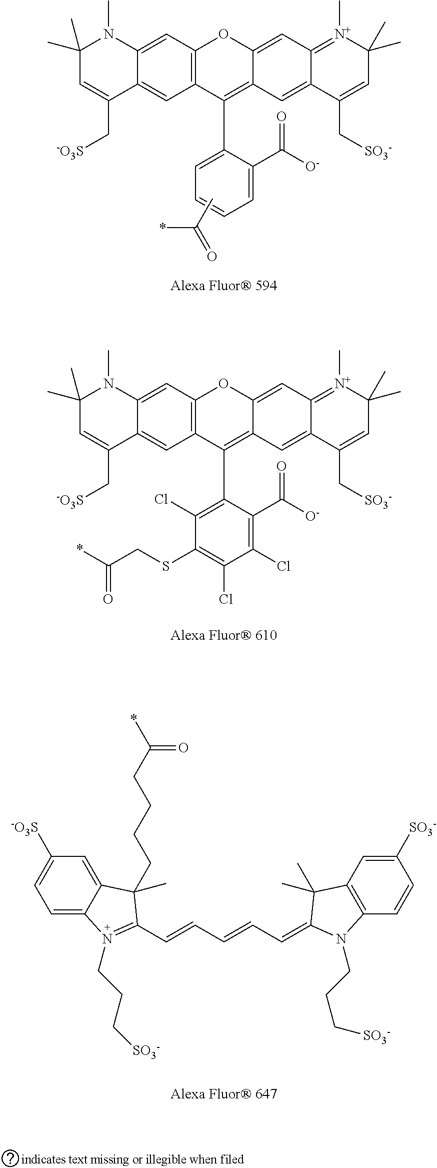

Illustrative radioactive molecules are those that are detectable by an imaging system, particularly in vivo imaging. Illustrative radioactive molecules include carbon-11, chromium-51, a fluorine agent (e.g., F-18 fluorodeoxyglucose, F-18 sodium fluoride, F-18 trifluoroacetic anhydride), a gallium agent (e.g., Ga-67 citrate, Ga-68 (e.g., Ga-68 DOTATATE), an iodine agent (e.g., I-123 or I-131), an indium agent (In-111), krypton-81m, nitrogen-13 oxygen-15, phosphorus-32, selenium-75, a technetium agent (e.g., technetium-99m), thallium-210 chloride, xenon-127, xenon-133, or a combination thereof.

In certain embodiments, the above-described diagnostic block co-polymer labeled with the complexing agent, e.g., labeled RNA, or by direct conjugation, the labeled diagnostic polyplex micelle nanoparticle is formed by complexing the labeled block co-polymer with scrambled, non-therapeutically active RNA. Upon determination of pancreatic disease, the patient can be treated using the same unlabeled polyplex micelle nanoparticle platform, in which the scrambled RNA is substituted with therapeutically active siRNA, i.e., gastrin, mutant KRAS and/or wild type KRAS. Illustrative examples of such therapeutic polyplex micelle nanoparticle platform are disclosed in U.S. Patent Application Publication US 2019/0076457, which is incorporated herein by reference. Illustrative scrambled RNA, and therapeutically active siRNAs are shown below:

gastrin siRNA
(SEQ ID NO: 4)
(si286 GUGCUGAGGAUGAGAACUA
(SEQ ID NO: 5)
GAUGCACCCUUAGGUACAG
(SEQ ID NO: 6)
AGAAGAAGCCUAUGGAUGG

### Sense and Antisense

Wildtype KRAS:
(SEQ ID NO: 7)
5′-GUUGGAGCUGGUGGCGUAG-3′
(SEQ ID NO: 8)
/5′-CUACGCCACCAGCUCCAAC-3′
Mutant KRAS:
(SEQ ID NO: 9)
5′-GUUGGAGCUGAUGGCGUAG-3′
(SEQ ID NO: 10)
/5′-CUACGCCAUCAGCUCCAAC-3′
Scrambled Control:
(SEQ ID NO: 11)
5′-CGAAGUGUGUGUGUGUGGCC-3′
(SEQ ID NO: 12)
/5′-GGCCACACACACACACUUCG-3′

The therapeutic polyplex micelle nanoparticles deliver siRNA using a CCK-receptor-targeted polyethylene glycol-block-poly(L-lysine) (PEG-b-PLL) polyplex. The targeted PEG-b-PLL polyplexes was designed to contain three basic features: (i) a short cationic segment (PLL) for the complexation of siRNA, (ii) a hydrophilic and biologically inert segment (PEG), and (iii) a cell surface targeting moiety (a peptide, gastrin-10). This block copolymer design will facilitate small polyplex formation following electrostatic interaction between the cationic polylysine moiety and negatively charged siRNA, resulting in charge neutralization and self-assembly into a polyplex structure with siRNA contained in the core surrounded by PEG conjugated to the targeting ligand gastrin-10 on the surface (FIG. 7). In certain embodiments, the polyplex disclosed herein is in the form of a micelle. The conjugation of gastrin-10 to the PEG-b-PLL polymer is performed via maleimide-thiol coupling chemistry.

In particular, the block copolymer includes two block moieties: (1) thiol-functionalized polyethylene glycol (PEG); and (2) poly(L-lysine) (PLL). The block copolymer (referred to herein as “SH-PEG-PLL”) may have a structure represented by

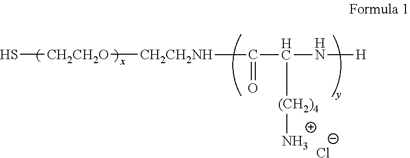

wherein x is 22 to 454, more particularly 45 to 275; and y is 10 to 100, more particularly 20 to 50. The number-average molecular weight of the PEG may range from 1000 Da to 20,000 Da. The number-average molecular weight of the PLL may range from 1600 Da to 16000 Da. In certain embodiments, x is 113 and y is 27, and the PEG molecular weight is 5000 g/mole (Da) and the PLL molecular weight is 4400 Da. In certain embodiments, 10 to 30%, more particularly about 20% of the PEG chains are thiol functionalized.

The maleimide-containing gastrin-10 peptide may have a structure of:

3-maleimido-propionyl-Glu-Glu-Glu-Ala-Tyr-Gly-Trp-Met-Asp-Phe-NH₂ (molecular formula: C₆₅H₇₉N₁₃O₂₂S; molecular weight: 1426.48 Da).
The maleimide-containing gastrin-10 peptide can be conjugated to any thiol (—SH) group through Michael addition to form a stable thioether bond.

The resulting nanoparticle has a structure of:

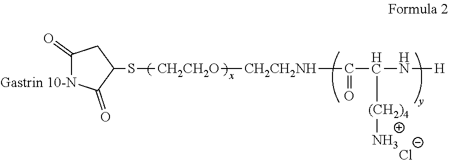

wherein x and y are the same as above.

In certain embodiments, the nanoparticle construct together has a structure of:

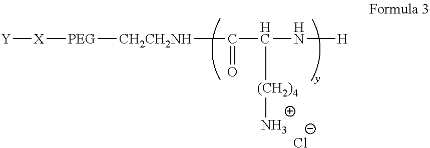

wherein Y is the cholecystokinin-B (CCK-B) receptor ligand; X is a linker; PEG is polyethylene glycol; and y is 10 to 200, more particularly 20 to 50.

The linker X may be a thioether or a group derived from a methoxy or carboxy linking agent.

At least one siRNA is mixed with the nanoparticle under conditions sufficient for electrostatically complexing the siRNA with the poly(L-lysine) of the polymer moiety. For example, GASTRIN-targeted siRNA and/or mutant KRAS-targeted siRNA may be added to the block copolymer construct by electrostatic complexation. The relative concentrations of the nanoparticle and the siRNA may vary. In certain embodiments, the relative concentrations are appropriate to provide N/P (nitrogen of polylysine amine (NH₂⁺) verses phosphate (PO₄⁻) of siRNA) of 0.5 to 20, more particularly 2 to 10.

In certain embodiments, the siRNA may be gastrin siRNA (si286

(SEQ ID NO: 4)
GUGCUGAGGAUGAGAACUA,
(SEQ ID NO: 5)
GAUGCACCCUUAGGUACAG
or
(SEQ ID NO: 6)
AGAAGAAGCCUAUGGAUGG

The cholecystokinin-B (CCK-B) receptor ligand may be gastrin-10 or a DNA aptamer as disclosed, for example, in Nucleic Acid Ther. 2017 Feb. 1; 27(1):23-35). An illustrative DNA aptamer has a structure of:

(SEQ. ID No: 3)
CATGGTGCAG GTGTGGCTGG GATTCATTTG CCGGTGCTGG
TGCGTCCGCG GCCGCTAATC CTGTTC.

### Compositions

In some embodiments, the methods disclosed herein involve administering to a subject in need of evaluation a composition, for example a composition that includes a pharmaceutically acceptable carrier and a diagnostic effective amount of one or more of the constructs disclosed herein. The subject may be suspected of having, or is at risk of having, pancreatic ductal adenocarcinoma (PDAC). In certain embodiments, the construct is administered for detecting the presence of a pancreatic intraepithelial neoplasia lesion.

The constructs, or compositions containing the construct, may be administered parenterally (including subcutaneous injections (SC or depo-SC), intravenous (IV), intramuscular (IM or depo-IM), intrasternal injection or infusion techniques), sublingually, intranasally (inhalation), intrathecally, topically, ophthalmically, or rectally. The composition may be administered in dosage unit formulations containing conventional non-toxic pharmaceutically acceptable carriers, adjuvants, and/or vehicles. The constructs are preferably formulated into suitable pharmaceutical preparations such as tablets, capsules, or elixirs for oral administration or in sterile solutions or suspensions for parenteral administration. Typically the constructs described above are formulated into compositions using techniques and procedures well known in the art.

For example, the compositions may be in a dosage unit form such as an injectable fluid, a nasal delivery fluid (e.g., for delivery as an aerosol or vapor), a semisolid form (e.g., a topical cream), or a solid form such as powder, pill, tablet, or capsule forms.

In some embodiments, one or more of the disclosed constructs are mixed or combined with a suitable pharmaceutically acceptable carrier to prepare a diagnostic composition. Pharmaceutical carriers or vehicles suitable for administration of the constructs provided herein include any such carriers known to be suitable for the particular mode of administration. Remington: The Science and Practice of Pharmacy, The University of the Sciences in Philadelphia, Editor, Lippincott, Williams, & Wilkins, Philadelphia, PA, 21^{st} Edition (2005), describes exemplary compositions and formulations suitable for delivery of the constructs disclosed herein.

Upon mixing or addition of the construct(s) to a pharmaceutically acceptable carrier, the resulting mixture may be a solution, suspension, emulsion, or the like. These may be prepared according to methods known to those skilled in the art. The form of the resulting mixture depends upon a number of factors, including the intended mode of administration and the solubility of the construct in the selected carrier or vehicle. Where the constructs exhibit insufficient solubility, methods for solubilizing may be used. Such methods are known and include, but are not limited to, using cosolvents such as dimethylsulfoxide (DMSO), using surfactants such as Tween®, and dissolution in aqueous sodium bicarbonate. Derivatives of the constructs, such as salts may also be used in formulating effective compositions. The disclosed constructs may also be prepared with carriers that protect them against rapid elimination from the body, such as time-release formulations or coatings. Such carriers include controlled release formulations, such as, but not limited to, microencapsulated delivery systems.

The construct is included in the pharmaceutically acceptable carrier in an amount sufficient to exert a diagnostically useful effect in the absence of undesirable side effects on the subject treated.

In some examples, about 0.1 mg to 1000 mg of a disclosed construct, a mixture of such construct, or a physiologically acceptable salt or ester thereof, is compounded with a physiologically acceptable vehicle, carrier, excipient, binder, preservative, stabilizer, flavor, etc., in a unit dosage form. The term “unit dosage form” refers to physically discrete units suitable as unitary dosages for human subjects and other mammals, each unit containing a predetermined quantity of active material calculated to produce the desired therapeutic effect, in association with a suitable pharmaceutical excipient. In some examples, the compositions are formulated in a unit dosage form, each dosage containing from about 1 mg to about 1000 mg (for example, about 2 mg to about 500 mg, about 5 mg to 50 mg, about 10 mg to 100 mg, or about 25 mg to 75 mg) of the one or more constructs. In other examples, the unit dosage form includes about 0.1 mg, about 1 mg, about 5 mg, about 10 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 400 mg, about 500 mg, about 600 mg, about 700 mg, about 800 mg, about 900 mg, about 1000 mg, or more of the disclosed construct(s).

The disclosed constructs or compositions may be administered as a single dose, or may be divided into a number of smaller doses to be administered at intervals of time.

Injectable solutions or suspensions may also be formulated, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid. Solutions or suspensions used for parenteral, intradermal, or subcutaneous application can include any of the following components: a sterile diluent such as water for injection, saline solution, fixed oil, a naturally occurring vegetable oil such as sesame oil, coconut oil, peanut oil, cottonseed oil, and the like, or a synthetic fatty vehicle such as ethyl oleate, and the like, polyethylene glycol, glycerine, propylene glycol, or other synthetic solvent; antimicrobial agents such as benzyl alcohol and methyl parabens; antioxidants such as ascorbic acid and sodium bisulfite; chelating agents such as ethylenediaminetetraacetic acid (EDTA); buffers such as acetates, citrates, and phosphates; and agents for the adjustment of tonicity such as sodium chloride and dextrose. Parenteral preparations can be enclosed in ampoules, disposable syringes, or multiple dose vials made of glass, plastic, or other suitable material. Buffers, preservatives, antioxidants, and the like can be incorporated as required.

Where administered intravenously, suitable carriers include physiological saline, phosphate buffered saline (PBS), and solutions containing thickening and solubilizing agents such as glucose, polyethylene glycol, polypropyleneglycol, and mixtures thereof. Liposomal suspensions including tissue-targeted liposomes may also be suitable as pharmaceutically acceptable carriers.

The constructs can be administered parenterally, for example, by IV, IM, depo-IM, SC, or depo-SC. When administered parenterally, a therapeutically effective amount of about 0.1 to about 500 mg/day (such as about 1 mg/day to about 100 mg/day, or about 5 mg/day to about 50 mg/day) may be delivered. When a depot formulation is used for injection once a month or once every two weeks, the dose may be about 0.1 mg/day to about 100 mg/day, or a monthly dose of from about 3 mg to about 3000 mg.

The constructs can also be administered sublingually. When given sublingually, the constructs should be given one to four times daily in the amounts described above for IM administration.

The constructs can also be administered intranasally. When given by this route, the appropriate dosage forms are a nasal spray or dry powder. The dosage of the constructs for intranasal administration is the amount described above for IM administration. When administered by nasal aerosol or inhalation, these compositions may be prepared according to techniques well known in the art of pharmaceutical formulation and may be prepared as solutions in saline, employing benzyl alcohol or other suitable preservatives, absorption promoters to enhance bioavailability, fluorocarbons, and/or other solubilizing or dispersing agents.

The constructs can be administered intrathecally. When given by this route, the appropriate dosage form can be a parenteral dosage form. The dosage of the compounds for intrathecal administration is the amount described above for IM administration.

### EXAMPLES

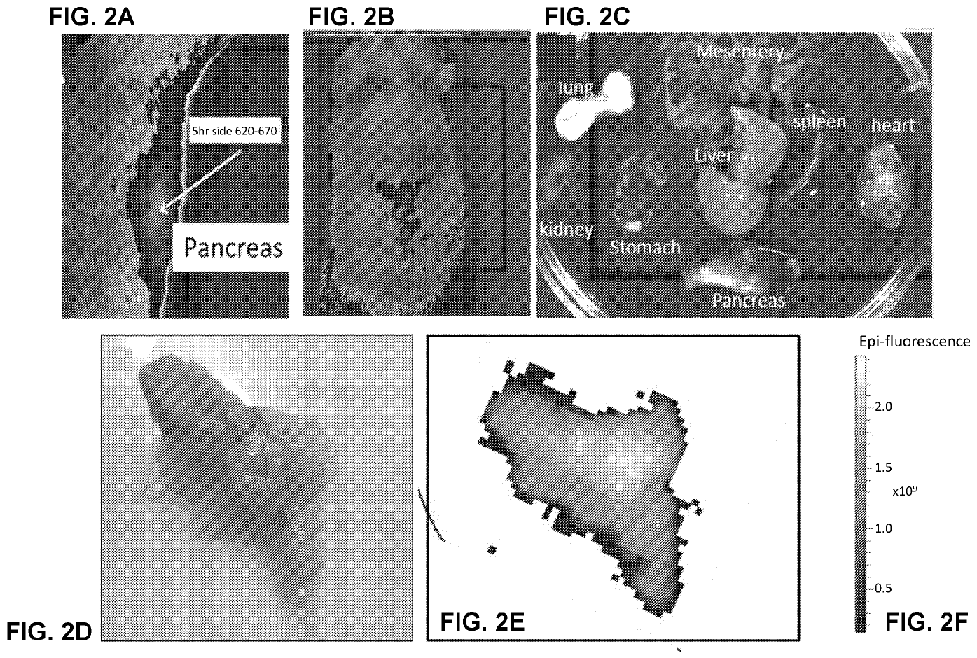

All animal studies were performed in an ethical fashion under a protocol approved by the Georgetown University IACUC. LSL-Kras^{G12D/+}; P48-Cre (KC) mice were bred and genotyped, and nanoparticle injections were performed via tail vein after the mice reached 5 month or 10 months of age, a time when PanIN lesions are well established. The peak fluorescent intensity in pancreas was reached 5 hours after injection and no fluorescence was detected 24 hours after injection. A representative 5 month-old anesthetized mouse is shown at 5 hours post injection (FIGS. 2A and 2B) in the IVIS imaging system demonstrating fluorescence in the near-infrared range consistent with uptake of the Alexa Fluor™ 647-labeled polyplex in the pancreas. Therefore, this 5 hour time point was selected to ethically euthanize the mice and collect the pancreas and other organs in order to examine ex-vivo fluorescence. Only the pancreas demonstrated positive fluorescence ex-vivo in comparison to the other organs (FIG. 2C). The excised pancreas was somewhat nodular and deformed due to the advancement of PanIN lesions and possible development of early pancreatic cancer (FIG. 2D). The same pancreas imaged separately ex-vivo in the IVIS system shows high intensity of fluorescence in the nodular region (FIG. 2E). The epi-fluorescent imaging scale is shown in FIG. 2F.

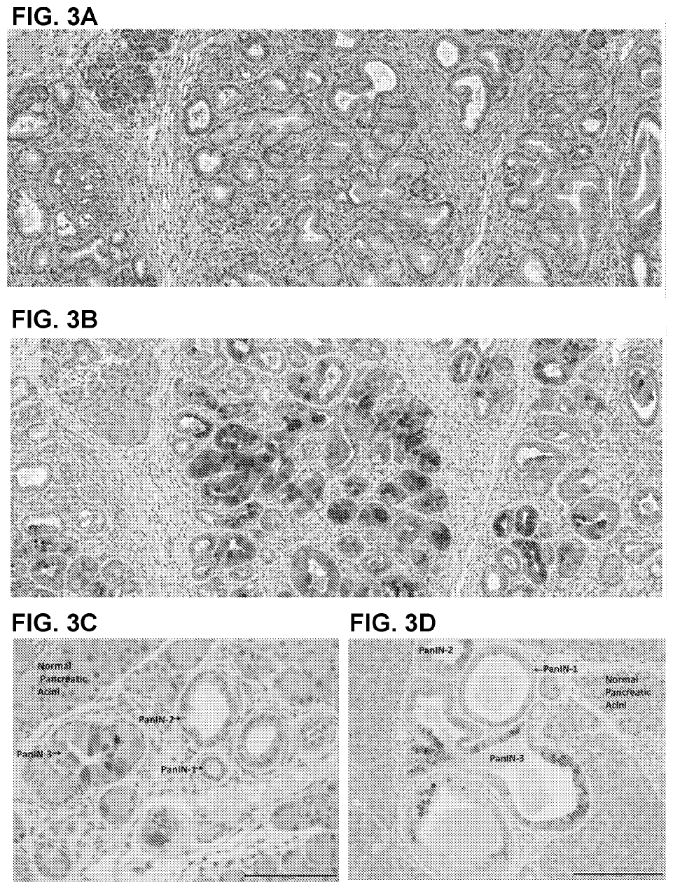

In order to confirm that the fluorescent-labeled targeted polyplex micelle indeed targeted CCK-BRs localized in the pancreatic PanIN lesions, and that there was limited off-target uptake in other organs, excised tissues were fixed and paraffin-embedded. Tissue sections from the pancreas and other major organs were examined by immunohistochemistry (IHC) with a selective rabbit anti-Alexa488 antibody (1:400; Cat. No. A-11094, Invitrogen) and then visualized with Rabbit Polink-1 horseradish peroxidase (HRP) staining. Hematoxylin & eosin (H&E) staining revealed the characteristic pancreatic histology, with advanced PanIN lesions and surrounding fibrosis, that occurs during pancreatic carcinogenesis (FIG. 3A). Confirmation that the CCK-BR targeted polyplex micelle distributed to the high grade PanIN lesions is demonstrated by immunohistochemistry of the serial pancreatic tissue section stained with a selective Alexa-488 antibody (FIG. 3B). Immunoreactivity was more intense in the PanIN-3 lesions with minimal staining in earlier stage PanIN-1b and PanIN 2 lesions and absence of staining in the normal pancreatic acinar cells (FIGS. 3C and 3D). These findings are confirmatory evidence that the fluorescence observed in the anesthetized mice by the IVIS imaging system upon polyplex micelle nanoparticle injection was indeed due to uptake of the nanoparticle in the CCK-BR expressing precancerous PanIN lesions of the mouse pancreas.

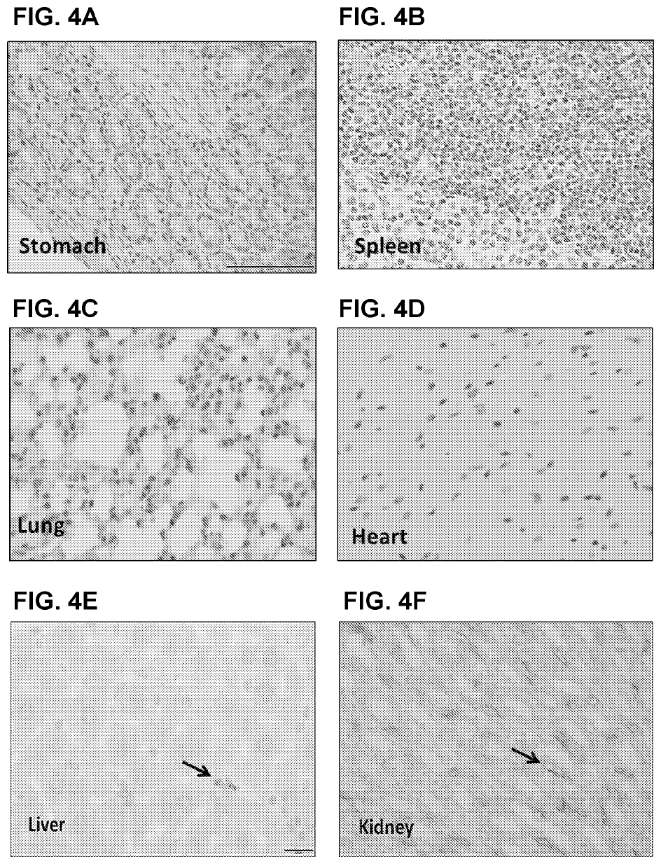

In order to confirm specificity and limited off-target uptake, other organs were also examined histologically and by anti-Alexa-488 immunohistochemical analysis. Immunohistochemistry was performed in sections of liver, kidney, spleen, heart, lung, and stomach (FIG. 4). Normal pancreas acinar tissue was evaluated compared to abnormal pancreas tissue containing PanIN lesions. Negative controls were run for each tissue, which involved replacing the primary anti-Alexa488 antibody with a nonspecific antibody reagent from the same host species and isotype (isotype control). Staining was considered specific when there was staining in the sample with no staining in the isotype control or control un-treated tissue. Slides were digitized with an Aperio ScanScope XT (Leica) at 200× in a single z-plane. Cell detection algorithms were run, and the staining intensity was scored using a scale of 0-3 as follows: 0 for no staining, 1 for mild staining, 2 for moderate, and 3 for strong staining. The percentage of cells staining positively are reported and an H-score, which integrates percent positive and staining intensity, was calculated using QuPath as follows:

$$H - score = [ 1 \times ( \% cells 1 + ) + 2 \times ( \% cells 2 + ) + 3 \times ( \% cells 3 + ) ] ; H - score range = 0 - 300.$$

The only significant staining was demonstrated in the PanIN lesions of the pancreas (Table 1).

TABLE 1
Alexa488 immunoreactivity scores in tissues. Three separate 5-month old mouse organs were scored for positive Alex-
488 antibody staining. The percentage of immunoreactive cells per tissue and the H-score shows only increased staining
in the PanIN lesions of the pancreas while the normal pancreas and other major organs are negative.

| | Normal Pancreas | | PanINs | | Liver | | Kidney | | Heart | | Stomach | |
|---|---|---|---|---|---|---|---|---|---|---|---|---|
| | % | | % | | % | | % | | % | | % | |
| Sample | positive | H-Score | positive | H-Score | positive | H-Score | positive | H-Score | positive | H-Score | positive | H-Score |
| ID98 | 0.01 | 0.01 | 9.33 | 21.82 | 0 | 0 | 0.02 | 0.02 | 0.06 | 0.07 | 0 | 0 |
| ID101 | 0 | 0 | 18.48 | 31.39 | 0.02 | 0.02 | 0.07 | 0.13 | 0 | 0.01 | 0 | 0 |
| ID114 | 0 | 0 | 31.72 | 58.32 | 0.01 | 0.01 | 0.01 | 0.01 | 0 | 0 | 0 | 0 |

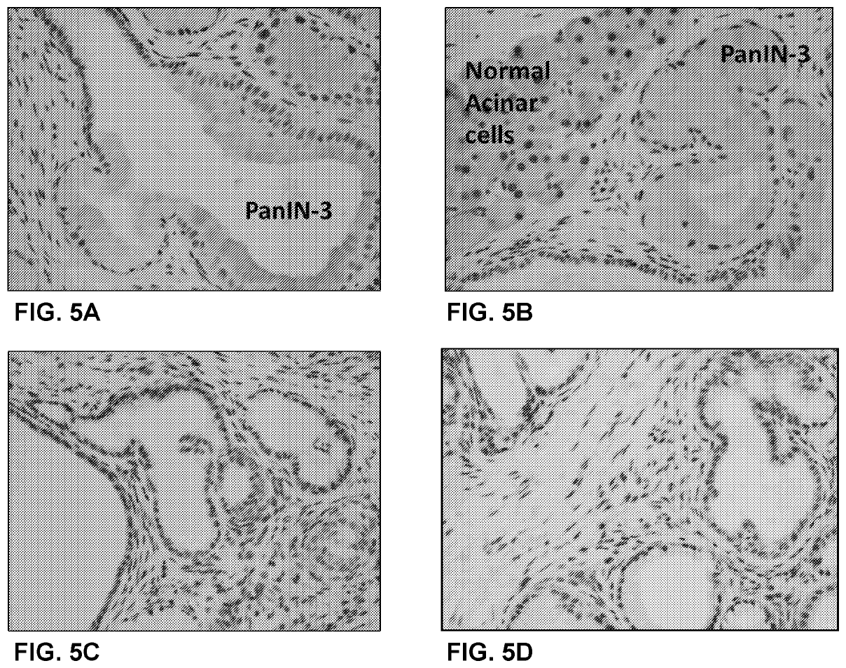

Immunoreactivity of the targeted polyplex nanoparticle localization in the pancreas PanINs of mice was also confirmed with an antibody to polyethylene glycol (PEG). Similar to the immunohistochemical localization with the Alexa488 antibody, the PEG antibody showed increased staining in the pancreas high grade PanIN lesions (FIGS. 5A and 5B). The normal pancreatic acinar cells that lack the CCK-BR did not exhibit any immunoreactivity to the PEG antibody (FIG. 5B). In contrast, age-matched KC mice injected with untargeted polyplex nanoparticles and reacted with the PEG antibody did not reveal any immunoreactivity in the pancreas PanIN lesions (FIGS. 5C and 5D). These findings are confirmatory evidence that the fluorescence observed in the anesthetized mice by the IVIS imaging system upon polyplex nanoparticle injection was indeed due to uptake of the nanoparticle in the CCK-BR expressing precancerous PanIN lesions of the mouse pancreas. Furthermore, these findings support the specificity of the CCK-BR targeted nanoparticle for PanIN uptake and localization and the lack of specificity with the untargeted nanoparticles. Since the CCK-BR target-specific NPs selectively bind to the high grade PanIN lesions in this KC mouse model, this tool could be developed for identification of precancerous lesions in the pancreas.

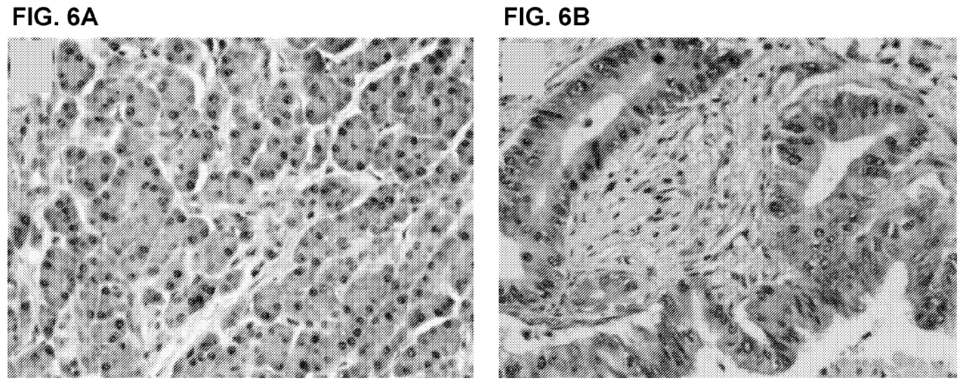

Human pancreas tissue using a tissue microarray were examined and demonstrated the absence of CCK-BR immunoreactivity in normal human pancreas (FIG. 6A), and the presence of CCK-BR immunoreactivity in human PanINs and pancreatic cancer (FIG. 6B).

The current recommendations today for PDAC screening includes high-risk individuals with a genetic predisposition or family history of PDAC. Although endoscopic ultrasound and MRI are used to monitor those subjects with cystic lesions of the pancreas, the overwhelming majority of cancers develop from non-cystic microscopic PanIN lesions that will require more sensitive imaging tools for detection. Although surgical resection offers a potential chance for a cure for pancreatic cancer if detected early, over 90% of the subjects have advanced disease at the time of presentation due the absence of sensitive accurate screening tests and biomarkers.

Although fluorophores were utilized for imaging in our murine model for proof of principle, more highly sensitive compounds have been used to enhance imaging in humans such as Gallium 68 (Ga-68) dotatate, a radiopharmaceutical tracer used for PET (positron emission tomography) imaging, or Technetium-99m used for CT-SPECT imaging. Utilizing the CCK-BR as a novel target for early detection of precancerous lesions in the pancreas may improve the sensitivity of imaging modalities and improve survival of pancreatic cancer by increasing the number of subjects with surgically resectable lesions.

### Materials and Methods

### Synthesis of the CCK-B Receptor-Targeted Polyplex

In order to develop the targeted NP, a thiol functionalized polyethylene glycol-block-poly(L-lysine)(SH-PEG-PLL) polymer was synthesized as previously described (Smith et al. Am. J. Physiol. 266, R277-R282-(1994)). To render the NP target-specific for the CCK-BR we used a maleimide linker to conjugate Gastrin-10 to the PEG via Michael addition reaction. The resulting Ga-PEG-PLL was extensively purified using a PD-10 column and dialysis. The polyplex micelle was prepared by mixing 1 mg/mL of the Ga-PEG-PLL with double stranded fluorophore labeled oligonucleotides Alexa Fluor™ 647 and 5′ Alexa Fluor™ 488 (Life Technologies, ThermoFisher Scientific) at an N/P ratio of 5. The nonspecific sequence of the Stealth Custom RNA, 5′ Alexa Fluor™ 647 was the following: Sense: AGCU-ACACUAUCGAGCAAUUAAC UU (SEQ. ID No: 1) and Anti-Sense: AAGUUAAUUGCUCGAUAGUGU-AGCU (SEQ. ID No: 2). The negative Control LOGC_a3N Custom RNA, 5′ Alexa Fluor™ 488 was proprietary but confirmed by NCBI Blast not to selectively inhibit and specific RNA.

### Breeding of LSL-Kras^{G12D/+}; P48-Cre (KC) Mice

All the animal research was performed under the approval by the Georgetown University IACUC. The mouse genomic KRAS locus upstream of a modified exon 1 engineered to contain a G>A transition in codon 12. This mutation, which is commonly found in human PDAC, expression of the mutated allele is achieved by interbreeding LSL-Kras^{G12D} mice with animals that express Cre recombinase from the pancreatic-specific promoter, P48. Heterozygote breeders (male or female) were mated. This Kras allele is non-functional in its germline configuration; therefore the mice are maintained by backcrossing heterozygous animals to C57BL/6. The usual litter size is approx. 8.

### Genotyping of LSL-Kras^{G12D/+}, P48-Cre (KC) Mice

At the time of weaning, mouse genotypes were determined by PCR analysis of tail DNA preparations. Tail biopsies were obtained from <3 week old mice with <0.5 cm removed after topical application of iced cold ethanol for anesthesia. This strain contains a point mutant allele of Kras, which is controlled by a Lox-Stop-Lox (LSL) cassette just upstream of exon 1. The genotyping strategy is to detect mice containing this LSL cassette. The genotyping protocol 1: Primer 005 is specific for the 3′ end of the LSL element, while primer 004 is specific for Kras exon 1. Primers 004 and 006 are used as an internal control to confirm the presence of amplifiable DNA. It is not possible to multiplex this reaction, since the 500 bp internal control amplicon dominates the reaction and quenches the production of the 550 bp mutant amplicon. The PCR product was visualized by gel electrophoresis.

Administration and Imaging of KC Mice and Organs with Polyplex Nanoparticle

Mice 5 and 10 months of age were used for the experiment. The rationale for these cohorts is that by age 5 months the mice with this LSL-Kras^{G12D/+}; P48-Cre genotype will have developed PanINs of all three stages (PanIN 1, 2, and 3) but not yet PDAC. By age 10 months early PDAC is often found histologically. One 10-month KC mouse, four 5-month old KC mice, and one 5-month old wild-type control mouse was used. Mice were anesthetized with isoflurane and a baseline image was obtained using the IVIS Lumina III in Vivo Optical Imaging System. Mice were injected with 0.1 ml of polyplex nanoparticle solution via tail vein. Images were obtained at the following time intervals after injection with the epi-fluorescent 620-670 filter: 20 minutes, 1 hour, 1.5 hours, 5 hours, and 24 hours. The peak fluorescence was found at 5 hours after injection and there was no further fluorescence after 24 hours. For controls, one age-matched wild type mouse was injected with the targeted polyplex nanoparticle and one age-matched 5 month old KC mouse was injected with non-targeted polyplex. All polyplex nanoparticles injected were complexed with the Alexa488 for IHC and Alexa647 for imaging.

Since 5 hours was identified as the optimal time for fluorescence after injection, this time was selected for harvesting of the pancreas and other major organs. At the 5 hour time point after injection of all study mice and controls, the organs were excised and ex-vivo organs were analyzed in the IVIS for fluorescence. Tissues were fixed with 4% paraformaldehyde at room temperature for 18-24 hours followed by paraffin embedding.

### Immunohistochemistry for Detection of Alexa488 Binding of Nanoparticles to Tissues

Immunohistochemistry to detect PLS-Alexa488 was performed using a biotin-free polymer approach (Golden Bridge International, Inc.) on 5-μm tissue sections mounted on Fisherbrand™ Superfrost™ Plus Microscope Slides (Thermo Fisher Scientific), which were dewaxed and rehydrated with double-distilled H2O. Heat induced epitope retrieval (HIER) was performed by heating sections in 0.01% citraconic anhydride containing 0.05% Tween-20 in a pressure cooker set at 122-125° C. for 30 s. Slides were incubated with blocking buffer (TBS with 0.05% Tween-20 and 0.25% casein) for 10 min then incubated with rabbit anti-Alexa488 antibody (1:400; Cat. No. A-11094, Invitrogen) diluted in blocking buffer over night at 4° C. Slides were washed in 1×TBS with 0.05% Tween-20 and endogenous peroxidases blocked using 1.5% (v/v) H2O2 in TBS (pH 7.4) for 10 min. Slides were incubated Rabbit Polink-1 HRP for 30 min at room temperature, washed and incubated with Impact™ DAB (3,3′-diaminobenzidine; Vector Laboratories) for 2-5 min. Slides were washed in ddH2O, counterstained with hematoxylin, mounted in Permount (Thermo Fisher Scientific), and whole tissue sections were scanned at high magnification (x200) using the ScanScope AT2 System (Aperio Technologies) yielding high-resolution data from the entire tissue section. Representative high-resolution images were extracted from these whole-tissue scans.

Slides were digitized with an Aperio ScanScope XT (Leica) at 200× in a single z-plane. Cell detection algorithms were run, and the staining intensity was scored using a scale of 0-3 as follows: 0 for no staining, 1 for mild staining, 2 for moderate, and 3 for strong staining. The percentage of cells staining positively are reported and an H-score, which integrates percent positive and staining intensity, was calculated using QuPath as follows:

$$H - score = [ 1 \times ( \% cells 1 + ) + 2 \times ( \% cells 2 + ) + 3 \times ( \% cells 3 + ) ] ; H - score range = 0 - 300.$$

### Immunohistochemistry for CCK-BR in Human Pancreas.

In order to investigate the clinical importance of our findings and the potential for this CCK-BR target specific polyplex nanoparticle to be used as an imaging tool for early detection of pancreatic cancer or high grade PanINs in human subjects, a pancreas tissue microarray (TMA) was obtained from US Biomax, (Cat. No. PA481). The human pancreas tissue microarray contained 48 unstained cores of fresh frozen paraffin embedded human pancreas tissues from both normal pancreas obtained at autopsy and from subjects with pancreatic adenocarcinoma. The tissue microarray was deparaffinized and subjected to antigen retrieval. The slide was washed in 1×PBS 3× for 2 minutes and then blocked using anti-goat HRP-DAB IHC Detection kit (cat #cts008-NOV, Novus Biologicals). The slide was incubated with the primary antibody CCK-BR (#Ab77077, Abcam) at 1:200 titer in PBS for overnight at 4° C. After rinsing, the slide was incubated with 1-3 drops of Biotinylated Secondary Antibody (Vial A, Novus) for 60 minutes. Then the slide was reacted with 1-3 drops of HSS-HRP (Vial B, Novus) for 30 minutes, washed and DAB Chromogen was added for 3 minutes.

In view of the many possible embodiments to which the principles of the disclosed invention may be applied, it should be recognized that the illustrated embodiments are only preferred examples and should not be taken as limiting the scope of the invention.

1. A method comprising detecting the presence of a pancreatic intraepithelial neoplasia lesion in a subject in vivo comprising administering to the subject a construct, or a pharmaceutically acceptable salt thereof, wherein the construct comprises:
(a) a polyethylene glycol-block-poly(L-lysine) polymer moiety, wherein the polyethylene glycol is thiol-functionalized;
(b) a cholecystokinin-B (CCK-B) receptor ligand coupled to the polyethylene glycol of the polymer moiety; and
(c) a detectable moiety complexed with, or conjugated to, the poly(L-lysine) of the polymer moiety,
wherein the construct is neutralized.

2. The method of claim 1, wherein the detectable moiety is a radioactive moiety.

3. The method of claim 2, wherein the radioactive moiety includes carbon-11, chromium-51, a fluorine agent, a gallium agent, an iodine agent, an indium agent, krypton-81m, nitrogen-13 oxygen-15, phosphorus-32, selenium-75, a technetium agent, thallium-210 chloride, xenon-127, xenon-133, or a combination thereof.

4. The method of claim 1, wherein the detectable moiety is Ga-68 DOTATATE or technetium-99mr.

5. The method of claim 1, wherein the detectable moiety is F-18 trifluoroacetic anhydride.

6. The method of claim 1, wherein the detectable moiety is a fluorophore.

7. The method of claim 6, wherein the fluorophore is a sulfonate dye.

8. The method of claim 7, wherein the sulfonate dye is a sulfonated coumarin, a sulfonated rhodamine, a sulfonated cyanine, or a sulfonated xanthene.

9. The method of claim 1, wherein the detectable moiety is complexed with the poly(L-lysine) of the polymer moiety.

10. The method of claim 1, wherein the detectable moiety is conjugated to the poly(L-lysine) of the polymer moiety.

11. The method of claim 1, wherein the cholecystokinin-B (CCK-B) receptor ligand comprises gastrin-10.

12. The method of claim 1, wherein the (a) and (b) moieties of the construct together have a structure of: 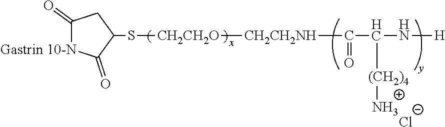
wherein x is 22 to 454, more particularly 45 to 275; and y is 10 to 200, more particularly 20 to 50.

13. The method of claim 1, wherein the (a) and (b) moieties of the construct together have a structure of: 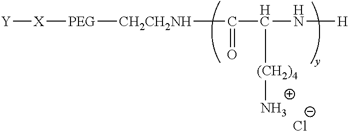
wherein Y is the cholecystokinin-B (CCK-B) receptor ligand; X is a linker; PEG is polyethylene glycol; and y is 10 to 200, more particularly 20 to 50.

14. The method of claim 1, wherein the construct comprises a polyplex structure having the detectable moiety in a core surrounded by the polyethylene glycol, wherein the cholecystokinin-B (CCK-B) receptor ligand is located on the surface of the polyplex structure.

15. The method of claim 1, wherein the method comprises administering the construct, or the pharmaceutically acceptable salt thereof to the subject, and then imaging the subject.

16. The method of claim 15, wherein the imaging detects the presence of a pancreatic intraepithelial neoplasia lesion in the subject.

17. The method of claim 1, wherein the subject is suspected of having, or is at risk of having, pancreatic ductal adenocarcinoma.

18. The method of claim 1, wherein the construct selectively targets cholecystokinin-B receptor over-expressed in the pancreatic intraepithelial neoplasia lesion.

19. The method of claim 1, wherein the cholecystokinin-B (CCK-B) receptor ligand binds to a cholecystokinin-B receptor over-expressed in the pancreatic intraepithelial neoplasia lesion.

20. The method of claim 1, wherein the detectable moiety comprises a detectable moiety tagged siRNA.

21. A method comprising:
(I) detecting the presence of a pancreatic intraepithelial neoplasia lesion in a subject in vivo comprising administering to the subject a first construct, or a pharmaceutically acceptable salt thereof, wherein the first construct comprises:
(a) a polyethylene glycol-block-poly(L-lysine) polymer moiety, wherein the polyethylene glycol is thiol-functionalized;
(b) a cholecystokinin-B (CCK-B) receptor ligand coupled to the polyethylene glycol of the polymer moiety; and
(c) a detectable moiety complexed with, or conjugated to, the poly(L-lysine) of the polymer moiety,
wherein the first construct is neutralized, and
(II) in a subject in which a pancreatic intraepithelial neoplasia lesion is detected, administering to the subject a therapeutically effective amount of a second construct, or a pharmaceutically acceptable salt thereof, comprising:
(a) a polyethylene glycol-block-poly(L-lysine) polymer moiety, wherein the polyethylene glycol is thiol-functionalized;
(b) a cholecystokinin-B (CCK-B) receptor ligand coupled to the polyethylene glycol of the polymer moiety; and
(c) a therapeutically active agent complexed with the poly(L-lysine) of the polymer moiety,
wherein the second construct is neutralized.


SEQUENCE LISTING (12 sequences)

SEQ ID NO: 1 - RNA, 25 nt, Artificial Sequence
  Synthetic oligonucleotide
agcuacacuaucgagcaauuaacuu

SEQ ID NO: 2 - RNA, 25 nt, Artificial Sequence
  Synthetic oligonucleotide
aaguuaauugcucgauaguguagcu

SEQ ID NO: 3 - DNA, 66 bp, Artificial Sequence
  Synthetic oligonucleotide
catggtgcaggtgtggctgggattcatttgccggtgctggtgcgtccgcggccgctaatcctgttc

SEQ ID NO: 4 - RNA, 19 nt, Artificial Sequence
  Synthetic oligonucleotide
gugcugaggaugagaacua

SEQ ID NO: 5 - RNA, 19 nt, Artificial Sequence
  Synthetic oligonucleotide
gaugcacccuuagguacag

SEQ ID NO: 6 - RNA, 19 nt, Artificial Sequence
  Synthetic oligonucleotide
agaagaagccuauggaugg

SEQ ID NO: 7 - RNA, 19 nt, Artificial Sequence
  Synthetic oligonucleotide
guuggagcugguggcguag

SEQ ID NO: 8 - RNA, 19 nt, Artificial Sequence
  Synthetic oligonucleotide
cuacgccaccagcuccaac

SEQ ID NO: 9 - RNA, 19 nt, Artificial Sequence
  Synthetic oligonucleotide
guuggagcugauggcguag

SEQ ID NO: 10 - RNA, 19 nt, Artificial Sequence
  Synthetic oligonucleotide
cuacgccaucagcuccaac

SEQ ID NO: 11 - RNA, 20 nt, Artificial Sequence
  Synthetic oligonucleotide
cgaaguguguguguguggcc

SEQ ID NO: 12 - RNA, 20 nt, Artificial Sequence
  Synthetic oligonucleotide
ggccacacacacacacuucg